(19) United States
(12) Patent Application Publication
(10) Pub. No.: US 2019/0021966 A1
(43) Pub. Date: Jan. 24, 2019

(54) WATER-SOLUBLE HYDROGEL-BASED DENTAL COMPOSITION AND METHODS OF MAKING AND USING SAME

(71) Applicant: DENTSPLY SIRONA INC., York, PA (US)

(72) Inventors: Amit JHA, Dover, DE (US); Thomas C. SIMONTON, Mount Wolf, PA (US)

(73) Assignee: DENTSPLY SIRONA Inc., York, PA (US)

(21) Appl. No.: 16/039,963

(22) Filed: Jul. 19, 2018

Related U.S. Application Data
(60) Provisional application No. 62/534,288, filed on Jul. 19, 2017.
(60) Provisional application No. 62/583,093, filed on Nov. 8, 2017.

(51) Int. Cl. A61K 8/41; A61K 8/69; A61K 8/21; A61K 8/73; A61K 8/81; A61K 8/04; A61K 8/58; A61K 8/49; A61K 8/23; A61Q 11/00

(52) U.S. Cl. CPC A61K 8/41; A61K 2800/5426; A61K 8/21; A61K 8/731; A61K 8/735; A61K 8/736; A61K 8/8147; A61K 8/8111; A61K 8/042; A61K 8/58; A61K 8/4986; A61K 8/23; A61Q 11/00; A61K 2800/48; A61K 2800/522; A61K 8/69

(57) ABSTRACT

Described herein are dental compositions, more particularly water soluble dental varnish composition useful for effective fluoridation, in situ biomimetic remineralization and improved adhesion to enamel. The dental composition includes a hydrogel-forming polymer having cohesive properties to itself and adhesive properties to a dental enamel. The hydrogel forming polymer includes a water-soluble polymer and an adhesion promotor chemically and/or physically conjugated to the water-soluble polymer. The embodiments also provide methods of forming a hydrogel forming polymer and use of hydrogel forming polymer to prepare such dental composition.

### FIELD OF THE DISCLOSURE

The present embodiments are directed to dental compositions, more particularly water soluble dental varnish composition useful for effective fluoridation, in situ biomimetic remineralization and improved adhesion to enamel. The dental composition includes a hydrogel-forming polymer having cohesive properties to itself and adhesive properties to a dental enamel. The hydrogel forming polymer includes a water-soluble polymer and an adhesion promotor chemically and/or physically conjugated to the water-soluble polymer. The embodiments also provide methods of forming a hydrogel forming polymer and use of hydrogel forming polymer to prepare such dental composition.

### BACKGROUND

Fluoride varnishes are applied to teeth to provide a prolonged source of fluoride ion to the tooth enamel so as to form a protective layer of calcium fluoride (CaF₂) on the tooth enamel and convert a portion of the hydroxyapatite to fluorapatite directly. Under physiological pH, the CaF₂ layer is insoluble and remains on the tooth, but the acid produced after carbohydrate intake and bacterial metabolism causes release of fluoride and calcium ions. The released fluoride ions may remain in the saliva or settle in free spaces on the tooth enamel and cavities. In particular, the fluoride ion is more electronegative than the hydroxide ion and reacts with the hydroxyapatite {[Ca₃(PO₄)₂]₃.Ca(OH)₂} of the tooth enamel to convert it to fluorapatite {[Ca₃(PO₄)₂]₃.CaF₂}. Thus, the formation of an acid-resistant layer of fluorapatite on the tooth surface can prevent tooth decay.

Conventional varnishes may include a natural resin as a tackifier, a synthetic polymer resin for film formation, a fluoride agent for fluoride release, an organic solvent to dissolve the resin, and additives to give the varnish a flavor or color. Natural resins may include, but are not limited to, rosin, rosin derivatives, mastic, or shellac. Synthetic polymer resins may include, but are not limited to, polyvinyl acetate (PVA), polyurethane methacrylate, or polyisocyanate. Such rosin/resin coatings tend to be hydrophobic and may not release sufficient fluoride in an effective manner. Fluoride agents may include, but are not limited to, sodium fluoride, stannous fluoride, acidulated phosphate fluoride, or fluorosilane. Additives may include, but are not limited to, titanium dioxide or sweeteners.

Solvents may include, but are not limited to, ethyl alcohol, isopropanol, ethyl acetate, butyl acetate, isoamyl propionate, hexane, or heptane. Solvents such as hexane or heptane, which may be effective for dissolving the resin/rosin, are not very biocompatible. Some conventional fluoride varnishes contain polymers dissolved in a solvent such as ethyl acetate or butyl acetate, which may be harsh on oral tissue and barely tolerable by the patient.

Sufficiently rapid adhesion between the varnish composition and the surface of the tooth ensures efficient delivery and maintenance of the varnish at the tooth surface.

Many conventional fluoride varnishes leave a long lasting hard coat on the teeth that must be broken and picked from the teeth. Moreover, many conventional fluoride varnishes may have a yellow color or other properties that make them not aesthetically pleasing to the patient.

### SUMMARY

There is continuing need for a varnish composition that overcomes the problems of existing varnish composition.

It is an object of the present disclosure to provide compositions that includes a hydrogel-forming polymer for efficient delivery and maintenance of the varnish at the tooth surface as well as that prevents discoloration of a hydrogel-forming polymer during conjugation and in the final varnish composition.

In a first aspect of the present disclosure disclosed herein is a method of forming a hydrogel-forming polymer having cohesive properties to itself and adhesive properties to dental enamel that comprises conjugating an adhesion promoter to a water-soluble polymer in the presence of an antioxidant to form the hydrogel-forming polymer having cohesive properties to itself and adhesive properties to dental enamel.

In one embodiment of the method of forming hydrogel-forming polymer, the antioxidant prevents discoloration of the hydrogel-forming polymer having cohesive properties to itself and adhesive properties to dental enamel during the conjugating.

In a second aspect of the present disclosure disclosed herein is a hydrogel-forming polymer produced by a process comprising: conjugating an adhesion promoter to a water-soluble polymer in the presence of an antioxidant to form the hydrogel-forming polymer having cohesive properties to itself and adhesive properties to dental enamel, wherein the antioxidant prevents discoloration of the hydrogel-forming polymer during the conjugating.

In a third aspect of the present disclosure disclosed herein is a composition that includes a hydrogel-forming polymer having cohesive properties to itself and adhesive properties to dental enamel, and water. The hydrogel-forming polymer comprises a water-soluble polymer and an adhesion promoter chemically and/or physically conjugated to the water-soluble polymer.

In one embodiment of the composition, the hydrogel-forming polymer further includes at least one antioxidant.

In another embodiment of the composition, the composition further includes a metal ion source.

In one embodiment, a composition includes a hydrogel-forming polymer having cohesive properties to itself and adhesive properties to dental enamel, a stimulus moiety, and water.

In a fourth aspect of the present disclosed herein, is a dental composition that includes a hydrogel-forming polymer having cohesive properties to itself and adhesive properties to dental enamel, a fluoride agent, and water. The hydrogel-forming polymer includes a water-soluble polymer and dopamine chemically and/or physically conjugated to the water-soluble polymer. The conjugated dopamine on the water soluble polymer adheres to any calcium ion present on the enamel surface and also absorbs calcium ions from a surrounding medium to the hydrogel-forming polymer.

In one embodiment of the dental composition, the hydrogel-forming polymer further includes at least one antioxidant.

In another embodiment of the dental composition, the composition further includes a stimulus moiety.

In one embodiment of the dental composition, the stimulus moiety is a branched cationic polymer.

In a fifth aspect of the present disclosure disclosed herein is a method of preparing a water soluble dental composition; said method comprising:

(a) dissolving a hydrogel-forming polymer in water to form a hydrogel-forming polymer solution;
(b) dissolving an antioxidant and a metal ion source in water to form a first solution and adding a branched cationic polymer to the first solution to form a branched cationic polymer solution;
(c) adding the branched cationic polymer solution to the hydrogel-forming polymer solution to prepare a hydrogel-forming polymer/branched cationic polymer mixture;
(d) dissolving an antioxidant in water to form a second solution and adding the second solution to the hydrogel-forming polymer/branched cationic polymer mixture to form an antioxidant polymer mixture; and
(e) mixing the antioxidant polymer mixture with a fluoride agent to form the water soluble dental composition.

In one embodiment of the method of preparing water soluble dental composition, the branched cationic polymer is selected to increase an adhesion kinetic between the composition and dental enamel.

Other features and advantages of the present disclosure will be apparent from the following more detailed description, taken in conjunction with the accompanying drawings which illustrate, by way of example, the principles of the disclosure.

### BRIEF DESCRIPTION OF THE DRAWINGS

The patent or application file contains at least one drawing executed in color. Copies of the patent or patent application publication with color drawing(s) will be provided by the Office upon request and payment of the necessary fee.

FIG. 1 schematically shows a synthesis scheme to produce Hyaluronic acid (HA)-g-dopamine.

FIG. 2 schematically shows the reaction mechanism for the synthesis scheme of FIG. 1.

FIG. 3 shows a proton NMR spectrum for HA-g-dopamine.

FIG. 4 shows an enlarged view of the 6.0 to 8.0 ppm region of the proton NMR spectrum of FIG. 3.

FIG. 5 shows the UV spectrum of dopamine.

FIG. 6 shows a calibration curve for dopamine.

FIG. 7 shows the viscosity of HA-g-dopamine in water before and after addition of a hydroxyapatite.

FIG. 8 shows a photograph of a bovine tooth stained by Alcian blue dye after application of HA-g-dopamine having a weight average molecular weight of about 1500 kDa.

FIG. 9 shows a photograph of a bovine tooth stained by Alcian blue dye after application of HA-g-dopamine having a weight average molecular weight of about 700 kDa.

FIG. 10 shows a photograph of a bovine tooth stained by Alcian blue dye after application of HA-g-dopamine having a weight average molecular weight of about 350 kDa.

FIG. 11 shows a photograph of a bovine tooth stained by Alcian blue dye after application of HA-g-dopamine having a weight average molecular weight of about 100 kDa.

FIG. 12 shows a photograph of a Polyacrylic acid (PAA)-g-dopamine sample formed in the presence of an antioxidant.

FIG. 13 shows a photograph of a PAA-g-dopamine sample formed in the absence of an antioxidant.

FIG. 14 shows a proton NMR spectrum for PAA-g-dopamine.

FIG. 15 shows a photograph of a bovine tooth stained by neutral red dye after application of PAA-g-dopamine.

FIG. 16 shows a photograph of a bovine tooth with no applied varnish stained by neutral red dye.

FIG. 17 shows a photograph of a varnish composition on a glass slide incubated in deionized (DI) water for 30 minutes.

FIG. 18 shows a photograph of a varnish composition on a glass slide incubated in a calcium chloride solution for 1 minute.

FIG. 19 shows a photograph of a varnish composition on a glass slide incubated in a calcium chloride solution for 30 minutes.

FIG. 20 shows a control hydroxyapatite disc with no varnish after staining with Alcian blue.

FIG. 21 shows a hydroxyapatite disc with a varnish composition including no stimulus moiety after staining with Alcian blue.

FIG. 22 shows a hydroxyapatite disc with a varnish composition having a stimulus moiety after staining with Alcian blue.

FIG. 23 shows schematically a water-soluble hydrogel-based dental varnish being applied to a tooth.

Wherever possible, the same reference numbers will be used throughout the drawings to represent the same parts.

### DETAILED DESCRIPTION

Provided herein are methods of forming a hydrogel forming polymer, use of the hydrogel forming polymer to prepare water-soluble, hydrogel-based dental compositions and methods of making and using the same.

Embodiments of the present disclosure, for example, in comparison to concepts failing to include one or more of the features disclosed herein, include a biocompatible polymer, are soluble in water, are compatible with a non-toxic organic solvent, are water-based, do not result in resin crystallization, are non-flammable, require no mixing, rapidly adhere to the enamel surface of a tooth, form a film on the enamel surface of a tooth, allow better dissolution of fluorides, provide more rapid diffusion of the fluoride ions to the enamel, provide a higher uptake of fluoride ions to the enamel, have a lower viscosity, are white in color, are colorless, are transparent, have rapid adhesion kinetics to dental substrates, absorb calcium ions, may be applied without drying, or combinations thereof.

In some embodiments, the hydrogel-forming polymer comprises a water-soluble polymer and an adhesion promoter chemically and/or physically conjugated to the water-soluble polymer.

In a first aspect of the present disclosure disclosed herein is a method of forming a hydrogel-forming polymer having cohesive properties to itself and adhesive properties to dental enamel that comprises conjugating an adhesion promoter to a water-soluble polymer in the presence of an antioxidant to form the hydrogel-forming polymer having cohesive properties to itself and adhesive properties to dental enamel.

The water-soluble polymer may be any hydrogel-forming polymer with adhesive and cohesive properties or any polymer that is hydrogel-forming and has adhesive and cohesive properties when conjugated to an adhesion promoter. In some embodiments, the water-soluble polymer is a natural polymer. In some embodiments, the water-soluble polymer is a synthetic polymer. In some embodiments, the water-soluble polymer is biocompatible.

In various embodiments, the phrase “adhesion promotor conjugated to the water soluble polymer” may be used interchangeably with “water soluble polymer conjugated to the adhesion promotor”.

In some embodiments of the method of forming hydrogel-forming polymer, the water-soluble polymer contains at least one functional group selected from the group consisting of carboxylic acid, amine, hydrazide, thiol, acrylic, methacrylic, and acrylamide.

In one embodiment of the method of forming hydrogel-forming polymer, the water-soluble polymer contains carboxylic acid.

In some embodiments of the method of forming hydrogel-forming polymer, the water soluble polymer has a weight average molecular weight in a range of 1 kDa to about 4000 kDa; such as from about 100 kDa to about 1500 kDa.

Suitable water-soluble polymers may include, but are not limited to, hyaluronic acid (HA), polyacrylic acid (PAA), chitosan, hydroxypropyl methylcellulose (HPMC), a water-soluble polyethylene glycol (PEG)-modified polymer, a water-soluble PEG-crosslinked polymer (such as, for example, a bis-thiol PEG), a water-soluble or partially water-soluble modified rosin, or combinations thereof.

HA is a naturally-occurring, water-soluble polymer found in connective tissue, epithelial tissue, and neural tissue. More specifically, HA is a non-sulfated, anionic glycosaminoglycan (GAG). HA was used as a starting polymer for the conjugation of an adhesion promoter, because HA is highly biocompatible, biodegradable, and non-immunogenic and has shown anti-inflammatory, antioedematous, antioxidant, and antibacterial effects after the treatment of periodontal disease and during wound healing. Unlike rosins and synthetic resins, which are difficult to remove and are irritating to the gingiva, HA may be easily removed by brushing and/or self-degradation and is non-irritating and beneficial to the gingiva.

In some embodiments, the water soluble polymer is the polyacrylic acid (PAA).

In some embodiments of the method of forming hydrogel-forming polymer, the adhesion promotor may be any compound that promotes adhesion and cohesion of the hydrogel-forming polymer. In one embodiment, the adhesion promoter is a natural compound. In some embodiments, the adhesion promoter is a synthetic compound. In some embodiments, the adhesion promoter is biocompatible.

In some embodiments of the method of forming hydrogel-forming polymer, the adhesion promotor contains at least one functional group selected from the group consisting of amine, carboxylic acid, thiol, acrylic, methacrylic, and acrylamide group.

In some embodiments of the method of forming hydrogel-forming polymer, the adhesion promotor contains an amine group.

Suitable adhesion promotors may include, but are not limited to, dopamine, dopamine with a conjugated electron-withdrawing group conjugated at the 6-position on the dopamine aromatic ring, dopamine complexed to an electron-withdrawing group at the hydroxyl groups of the dopamine, gallic acid, caffeic acid, ferulic acid, protocatechuic acid, coumaric acid, ellagic acid, resveratrol, rosmarinic acid, quercetin, or combinations thereof. In some embodiments, the conjugated electron-withdrawing group is a nitro group (—NO₂), a chloro group (—Cl), or a fluoro group (—F). In some embodiments, the complexed electron-withdrawing group is a borate or a borate derivative.

Biomaterials in nature have precisely-controlled adhesiveness and cohesiveness properties. For example, mussel adhesive foot protein (Mafp), secreted by certain marine mussels, has dual adhesive and cohesive features that are controlled by a dopamine amino acid found in the protein. An adhesion promoter grafted water-soluble polymer with both adhesive and cohesive film formation properties provides effectiveness in a water-soluble dental varnish system in accordance with exemplary embodiments. The molecular basis for adhesion is the reversible coordination of metal oxides, π-π interactions with various synthetic polymers and irreversible covalent bonding to any surface. For cohesive function, catechol undergoes pH-dependent oxidative reactions by the dopamine-to-quinone transition. Thus, dopamine may promote both adhesion and cohesion.

In some embodiments, the adhesion promoter is a modified version of a naturally-occurring compound. The modification preferably improves the adhesive and cohesive properties and/or the stability of the adhesion promoter in the water-soluble hydrogel-based dental varnish.

In some embodiments of the method of forming hydrogel-forming polymer, conjugating the adhesion promoter to the water-soluble polymer occurs through an amidation reaction in an aqueous solution using carbodiimide catalysis system in the presence of a co-catalyst to form a reaction solution.

In some embodiments, the carbodiimide in carbodiimide catalysis system is 1-ethyl-3-(3-dimethylaminopropyl)carbodiimide (EDC).

In some embodiments, the co-catalyst is selected from the group consisting of hydroxybenzotriazole (HOBt), N-hydroxysuccinimide (NHS) and sulfo-N-hydroxysuccinimide (Sulfo-NHS).

In some embodiments of the method of forming hydrogel-forming polymer, the carbodiimide catalysis system is selected from the group consisting of 1-ethyl-3-(3-dimethylaminopropyl)carbodiimide (EDC)/hydroxybenzotriazole (HOBt), 1-ethyl-3-(3-dimethylaminopropyl)carbodiimide (EDC)/N-hydroxysuccinimide (NHS), and 1-ethyl-3-(3-dimethylaminopropyl)carbodiimide (EDC)/sulfo-N-hydroxysuccinimide (Sulfo-NHS).

In some embodiments of the method of forming hydrogel-forming polymer, the water soluble polymer is present at a concentration of from about 0.01 weight percent to about 50 weight percent based on total volume of the reaction solution, such as in the range of from about 0.1 weight percent to 20 weight percent or in the range of from about 1 weight percent to about 10 weight percent.

In some embodiments of the method of forming hydrogel-forming polymer, the adhesion promoter may be added in a 1:100 to 50:100 molar ratio with respect to the number of available functional groups on a repeating unit of the water-soluble polymer; such as 30:100 molar ratio with respect to the number of available functional groups on the repeating unit of the water-soluble polymer.

In some embodiments of the method of forming hydrogel-forming polymer, the adhesion promotor is present in concentration of from about 1 mole percent to about 80 mole percent based on repeating unit of functional groups on the water-soluble polymer.

In some embodiments, the 1-ethyl-3-(3-dimethylaminopropyl)carbodiimide (EDC) is added in the range of from 1 to 10 mmol per mol of available functional groups on a repeating unit of water-soluble polymer; alternatively in the range of 2 to 5 mmol, alternatively about 3 mmol, or any value, range, or sub-range there between, per mol of available functional groups on the repeating unit of water-soluble polymer.

In some embodiments, the co-catalyst is added in the range of from 1 to 10 mmol based on per mol of available functional groups on a repeating unit of water-soluble polymer; alternatively in the range of 2 to 5 mmol, alternatively about 3 mmol, or any value, range, or sub-range there between, per mol of available functional groups on the repeating unit of water-soluble polymer.

In some embodiments, the hydrogel forming polymer is HA-g-dopamine or PAA-g-dopamine.

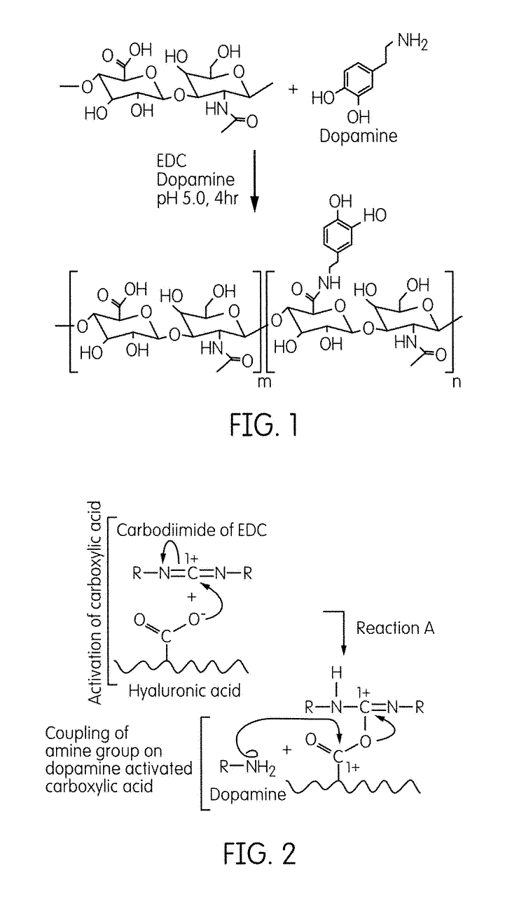

In some embodiments, HA-g-dopamine or PAA-g-dopamine were synthesized by the process described above. The synthesis scheme to produce HA-g-dopamine is shown in FIG. 1 and the mechanism of the EDC chemistry is shown in FIG. 2.

In some embodiments, HA-g-dopamine or PAA-g-dopamine were characterized by percentage of conjugation, molecular weight, and distribution.

In some embodiments, the percent conjugation of the adhesion promoter, for example, dopamine in the HA-g-dopamine or PAA-g-dopamine may be in a range of 5 to 80%. In one embodiment the percent conjugation of the dopamine in the HA-g-dopamine or PAA-g-dopamine is in the range of 20 to 30%.

Under certain conditions, including high pH or in the presence of an oxidant, the catechol group in dopamine oxidizes to a quinone group, which causes a black coloration during the synthesis of dopamine-grafted polymers. Formation of the quinone causing the black coloration may be prevented by adding an antioxidant during the synthesis.

In one embodiment, the antioxidant prevents discoloration of the hydrogel-forming polymer having cohesive properties and adhesive properties to dental enamel during the conjugating.

In some embodiments of the method of forming hydrogel-forming polymer, the antioxidant is selected from the group consisting of ascorbic acid, sodium metabisulfite, boric acid, sodium tetraborate, 4,4′-Biphenyldiboronic acid, benzene-1,4-diboronic acid, 2,5-thiophenediyl bisboronic acid, sulfur dioxide, uric acid, tocopherol and mixtures thereof.

Dopamine-grafted polyacrylic acid was synthesized in the presence of ascorbic acid, and no discoloration or black color formation was observed during or after the synthesis.

In some embodiments of the method of forming hydrogel-forming polymer, the antioxidant is present in the hydrogel-forming polymer in amounts of from 0.1 to 14 mmol based on per mol of available functional groups on a repeating unit of the water-soluble polymer; alternatively in the range of 0.5 to 10 mmol, alternatively in the range of 0.5 to 5 mmol, alternatively in the range of 1 to 5 mmol, or any value, range, or sub-range there between, per mmol of available functional groups on the repeating unit of the polymer.

In certain aspect of the present disclosure provided herein is a hydrogel-forming polymer produced by a process comprising: conjugating an adhesion promoter to a water-soluble polymer in the presence of an antioxidant to form the hydrogel-forming polymer having cohesive properties and adhesive properties to dental enamel, wherein the antioxidant prevents discoloration of the hydrogel-forming polymer during the conjugating.

### Dental Compositions

In certain aspect of the present disclosure, a composition includes a hydrogel-forming polymer with cohesive properties to itself and adhesive properties to dental enamel in water.

In some embodiments, the composition does not include any ethanol, iso propanol, ethyl acetate, butyl acetate, isoamyl propionate or hexane.

As discussed above, the hydrogel-forming polymer comprises a water-soluble polymer and an adhesion promoter chemically and/or physically conjugated to the water-soluble polymer.

In some embodiments of the composition, the adhesion promoter provides the hydrogel-forming polymer with cohesive properties to itself and adhesive properties to dental enamel.

In some embodiments of the composition, the hydrogel-forming polymer further includes at least one antioxidant.

In some embodiments of the composition, the composition includes a metal ion source. It will be understood that there is no particular limitation to the source of the metal ions.

In some embodiments of the composition, the metal ion source is selected from the group consisting of a divalent metal ion source, a trivalent metal ion source, and mixtures thereof.

Examples of suitable divalent metal ion sources include, but are not limited to, a salt of calcium, salt of zinc, salt of magnesium, salt of tin, salt of strontium, salt of chromium, salt of manganese, salt of beryllium, salt of barium, salt of cobalt, salt of nickel, salt of lead and salt of copper.

Examples of suitable trivalent metal ion sources include, but are not limited to, a salt of aluminum, salt of iron, salt of chromium, salt of bismuth, salt of manganese, salt of cobalt and salt of indium.

In some embodiments of the composition, the composition further comprises at least one stimulus moiety.

A stimulus moiety, as used herein, refers to any molecule or part of a molecule that increases the adhesion kinetics of the hydrogel-forming polymer having cohesive properties and adhesive properties to dental enamel in a dental composition upon inclusion in the dental composition. The stimulus moiety may be cationic, linear (unbranched) or branched, and non-polymeric or polymeric.

In some embodiments, the at least one stimulus moiety is a branched cationic polymer.

In some embodiments, the branched cationic polymer are included in the composition to improve the adhesion kinetics of the hydrogel-forming polymer having cohesive properties and adhesive properties to dental enamel.

Stimulus moieties may include, but are not limited to, lysine, arginine, polylysine, polyarginine, linear polyethyleneimine, branched polyethyleneimine, or poly(diallyldimethylammonium chloride) (polyDADMAC), or combinations thereof.

In some embodiments, the ratio of stimulus moiety cationic groups to hydrogen-forming polymer repeating unit functional groups is in a range of 1:2 to about 2:1, alternatively in the range of 1:2 to 1:1, alternatively in the range of 1:1 to 2:1, alternatively about 1:1, or any value, range, or sub-range there between.

Certain mussel foot proteins, such as, for example, mfp-3 and mfp-5, are rich in dopamine as well as the amino acid lysine, which is frequently in adjacent positions along the protein backbone. These proteins have impressive wet adhesion to mineral, oxide, and organic surfaces. The dopamine units in mfp-3 and mfp-5 form bidentate coordination and hydrogen bonds to mineral and oxide surfaces and hydrophobic interactions on polymeric surfaces, but only if protected from oxidation in a low pH environment and in the presence of antioxidant during deposition. Further, the lysine being present in positions adjacent to the dopamine serves as a stimulus moiety to further enhance the adhesion by disrupting the hydration layer formed by water on the polar surfaces.

The presence of alkyl ammonium functionalities, such as, for example, in the amino acids lysine and 2,4-diaminobutyric acid (Dab), in catecholic polymers or compounds limits the oxidation, and the amine and catechol moieties may interact synergistically to mediate surface priming by the catechol alkylamine compounds to mineral surfaces and promote higher adhesion to surfaces. Increasing the ratio of cationic amines to catechols in a molecule reduces adhesion, and the catechol-cation synergy is greatest when both functionalities are present within the same molecule.

In some embodiments, polyethylene imine, a branched cationic polymer, was selected to provide stimulus moieties and was mixed with a dopamine-grafted polymer to have amine and catechol functionalities together on same polymer network. The relative adhesion was tested for formulations prepared with and without the cationic polymer, which clearly demonstrated higher and rapid adhesion of the polymer network containing branched cationic polymer.

In certain aspect of the present disclosure, a water-soluble dental varnish is provided that includes a hydrogel-forming polymer with cohesive properties to itself and adhesive properties to dental enamel. In some embodiments, the cohesive and adhesive properties are provided by an adhesion promoter that is conjugated chemically and/or physically to a water-soluble polymer to provide the hydrogel-forming polymer. The hydrogel-forming polymer is dissolved in water with a fluoride agent to form the water-soluble hydrogel-based dental varnish.

In some embodiments, the primary solvent in the water-soluble hydrogel-based dental varnish is water.

In some embodiments, the only solvent in the water-soluble hydrogel-based dental varnish is water.

In some embodiments, the water-soluble hydrogel-based dental varnish does not include any ethanol, iso propanol, ethyl acetate, butyl acetate, isoamyl propionate or hexane.

In some embodiments, the water-soluble hydrogel-based dental varnish is free of rosins or substantially free of rosins.

In some embodiments, the water-soluble dental varnish further includes a metal ion source as described above.

In some embodiments, the water-soluble dental varnish further includes a stimulus moiety as described above.

In certain embodiments of the water-soluble dental varnish composition disclosed herein, the fluoride agent is selected from the group consisting of sodium fluoride, stannous fluoride, acidulated phosphate fluoride, amine fluoride, fluorosilane and mixture thereof.

In some embodiments, the amine fluoride is selected from the group consisting of N′,N′-tri-(polyoxyethylene)-N-hexadecylpropylene diamine dihydrofluoride; 9-octadecylamine hydrofluoride, hexadecylamine hydrofluoride and bis-(hydroxyethyl)-aminopropyl-N-hydroxyethyloctadecylamine dihydrofluoride.

In certain embodiments of the water-soluble dental varnish composition disclosed herein, the fluoride source is present in a concentration of from about 0.01 weight percent to about 10 weight percent based on a total weight of the composition; such as in the range of from about 1 weight percent to about 8 weight percent or in the range of from about 2 weight percent to about 7 weight percent.

In certain embodiments of the dental varnish composition disclosed herein, the dental varnish releases fluoride ions in a concentration ranging from 1000 ppm to 22600 ppm.

The fluoride ion source may be in an amount such that it is capable of providing a high level of fluoride ion in the composition, that is at least about 1,000 ppm, and in some instances up to as much as 30,000 ppm, e.g., from about 7,000 ppm to about 27,000 ppm, from about 15,000 ppm to about 25,000 ppm, or about 22,000 or 23,000 ppm. In order to provide such a concentration in the optimal ppm range, the exact weight percentage of the fluoride ion source in the composition may vary, depending upon the stoichiometric properties of different fluoride ion sources.

In certain embodiments of the water-soluble dental varnish composition disclosed herein, the hydrogel-forming polymer is present in a concentration of from about 0.01 weight percent to about 50 weight percent based on a total volume of the composition; such as in the range of from about 0.1 weight percent to 20 weight percent or in the range of from about 1 weight percent to about 10 weight percent.

In some embodiments, the water-soluble dental varnish also includes one or more of a thickener, a tackifier, a flavoring agent, a sweetener, and a colorant.

Examples of thickener include, but are not limited to fumed silica, carboxyvinyl polymers, carrageenans, karaya, gum arabic and tragacanth, magnesium aluminum silicate. The amount of thickener present in the dental varnish in amounts of from about 0.1 weight percent to about 1.0 weight percent, such as from about 0.5 weight percent to about 5.0 weight percent or from about 1 weight percent to about 10 weight percent.

Examples of a tackifier suitable for use herein may include, but are not limited to rosin, mastic, shellac, cellulose and cellulose derivatives, pullulan, xanthan gum, gellan gum. Such tackifiers as described herein may be present in the dental varnish in amounts of from about 0.01 weight percent to about 0.1 weight percent, such as from about 0.05 weight percent to about 1 weight percent or from about 1 weight percent to about 10 weight percent.

Examples of a suitable flavoring agent include but are not limited to peppermint, watermelon, wintergreen, spearmint, cherry, citric acid, orange, strawberry, vanilla, coconut, bubble gum flavors and mixtures thereof. Such flavoring agents if present, may be in the dental varnish in amounts of from about 0.001 weight percent to about 0.05 weight percent, such as from about 0.005 weight percent to about 0.5 weight percent or from about 0.01 weight percent to about 5 weight percent.

Examples of a suitable sweetener include but not limited to xylitol, sorbitol, sucralose, aspartame, sodium saccharin, and mixtures thereof. Such sweeteners may be in the dental varnish in amounts of from about 0.001 weight percent to about 0.02 weight percent, such as from about 0.005 weight percent to about 0.2 weight percent or from about 0.01 weight percent to about 2.0 weight percent.

Examples of a suitable colorant may be caramel, beta-carotene, annatto or titanium dioxide. Such colorant may be in the dental varnish in amounts of from about 0 weight percent to about 2 weight percent, such as from about 0.01 weight percent to about 1.0 weight percent or from about 0.08 weight percent to about 1.0 weight percent.

In some embodiments, the dental varnish formulation is optimized for stability, cohesive and adhesive properties, fluoride loading and release kinetics, biocompatibility, and uptake of fluoride to enamel and remineralization. To further improve the gelation kinetic and mechanical properties, one or more stimuli-sensitive polymers, which may include, but are not limited to, chitosan and hydroxypropyl methylcellulose (HPMC), may be included in the final varnish formulation. In some embodiments, the stimuli-sensitive polymers are present in the range of 0.1 weight percent to 50 weight percent with respect to the volume of the hydrogel-forming polymer in the formulation.

Also, disclosed herein are methods of preparing a water soluble dental composition. The water soluble dental composition of the present disclosure may be prepared in general by
(a) dissolving a water soluble polymer in 75-200 mL water to form a water soluble polymer solution and adjusting the pH of the water soluble polymer solution to about 6.2-7.0;
(b) optionally adding 0.1 to 14 mmol of antioxidant to the water soluble polymer solution;
(c) adding 1-10 mmol of 1-ethyl-3-(3-dimethylaminopropyl)carbodiimide per mole of available functional group on a repeating unit of water soluble polymer and readjusting the pH water soluble polymer solution to about 6.8;
(d) adding 1-10 mmol co-catalyst per mole of available functional group on the repeating unit of water soluble polymer and readjusting the pH water soluble polymer solution to about 6.8
(e) adding adhesion promotor in 1:100 to 50:100 molar ratio with respect to number of available functional group on the repeating unit of water soluble polymer to form a hydrogel forming polymer reaction mixture;
(f) dialyzing and lyophilizing the hydrogel forming polymer reaction mixture to form hydrogel forming polymer;
(g) dissolving hydrogel-forming polymer in water to form a hydrogel-forming polymer solution;
(h) mixing the hydrogel-forming polymer solution with a fluoride agent to form the dental composition.

In one embodiment water soluble dental composition may be prepared by
(a) dissolving a hydrogel-forming polymer in water to form a hydrogel-forming polymer solution;
(b) dissolving an antioxidant and metal ion source in water to form a first solution;
(c) optionally adding a branched cationic polymer to the first solution to form a branched cationic polymer solution;
(d) adding the first solution to the hydrogel-forming polymer solution to form a hydrogel-forming polymer mixture; with the proviso that step (d) is skipped if said branched cationic polymer is added to the first solution;
(e) optionally adding the branched cationic polymer solution to the hydrogel-forming polymer solution to prepare a hydrogel-forming polymer/branched cationic polymer mixture;
(f) dissolving an antioxidant in water to form a second solution;
(g) optionally adding the second solution to the hydrogel-forming polymer/branched cationic polymer mixture to form an antioxidant polymer mixture;
(h) adding the second solution to the hydrogel-forming polymer mixture to form antioxidant hydrogel-forming polymer mixture; with the proviso that step (h) is skipped if the second solution is added to the hydrogel-forming polymer/branched cationic polymer mixture;
(i) optionally mixing the antioxidant polymer mixture with a fluoride agent to form the water soluble dental composition;
(j) mixing the antioxidant hydrogel-forming polymer mixture with a fluoride agent to form the water soluble dental composition with the proviso that step (j) is skipped if the antioxidant polymer mixture is mixed with the fluoride agent.

In certain embodiments of methods of preparing a water soluble composition, the branched cationic polymer is selected to increase an adhesion kinetic between the composition and dental enamel.

In certain embodiments of methods of preparing a water soluble composition as described herein, the hydrogel-forming polymer may be present in amounts of from about 0.01 weight percent to about 50 weight percent based on the total volume of composition such as from about 0.1 weight percent to about 20 weight percent or from about 1.0 weight percent to about 10 weight percent.

In certain embodiments of methods of preparing a water soluble composition, the antioxidant may be present in the dental composition in amounts of from about 0.005 mmole/ml to about 20 mmole/ml based on the total volume of hydrogel-forming polymer, such as from about 0.025 mmole/ml to about 10 mmole/ml or from about 0.05 mmole/ml to about 5 mmole/ml.

In certain embodiments of methods of preparing a water soluble composition, the metal ion source may be present in the dental composition in amounts of from about 0.001 mmole/ml to about 5 mmole/ml based on the total volume of hydrogel-forming polymer, such as from about 0.002 mmole/ml to about 1 mole/ml or from about 0.01 mmole/ml to about 2 mmole/ml.

In certain embodiments of methods of preparing a water soluble composition, the branched polyethylene imine having an average molecular weight of about 600 Da, 1200 Da, and 1800 Da was added to the solution of an antioxidant and metal ion source to form a form a branched cationic polymer solution.

In certain embodiments of methods of preparing a water soluble composition, the branched cationic polymer may be present in the dental composition in amounts of from about 0.1 weight percent to about 50 weight percent based on the total volume of composition, such as from about 0.5 weight percent to about 20 weight percent or from about 1 weight percent to about 10 weight percent.

In some embodiments, a method includes applying a composition to a dental surface. The composition includes a hydrogel-forming polymer having cohesive properties and adhesive properties to dental enamel, a fluoride agent, and water.

### Properties/Uses

In certain embodiments of the dental varnish composition disclosed herein, the hydrogel-forming polymer promoted increased adhesion of the varnish composition to a tooth surface.

The catechol moiety of dopamine-grafted polymers (e.g. polyacrylic acid-g-dopamine) has the capacity to bind strongly to certain metal ions, including calcium. Such a feature may accelerate remineralization of teeth by hydroxyapatite formation via co-precipitation of calcium and phosphate ions from saliva in situ. Thus, a dopamine-grafted polymer-based varnish system is not only capable of rapid fluoride release and uptake but may also promote in situ biomimetic remineralization by absorbing calcium from surrounding saliva. Conventional varnish systems do not have such an in situ biomimetic remineralization feature.

Although hydrogel-forming polymers having cohesive properties and adhesive properties to dental enamel have been described herein in water-soluble hydrogel-based dental varnishes, hydrogel-forming polymers having cohesive properties and adhesive properties to dental enamel may also be included in compositions for osseo-integration of dental implants, in compositions for repair of cracked teeth, in dental adhesive compositions, in medicament delivery systems, in remineralization compositions, or in paint-on strips with whitening agents.

The disclosure discussed herein is further illustrated by the compositions described in the following Examples, but these Examples should not be construed as limiting the scope of the present disclosure.

### Example-1 Synthesis of HA-g-Dopamine

300 mg of HA, having a weight average molecular weight of about 100 kDa, was dissolved in 100 mL of deionized (DI) water at room temperature over a period of 5 to 10 hours. The pH of the solution was adjusted to 5.5 with 0.1 N hydrochloric acid (HCl) and 0.1N sodium hydroxide (NaOH) aqueous solutions. Ascorbic acid was then added to the solution to achieve a concentration of 1 mg/mL, and dopamine was added in a 30:100 molar ratio with respect to the number of available carboxylic groups on the repeating unit of HA. The pH of this resulting solution mixture was then adjusted to about 6.8. Then, 3 mmol of EDC per mol of available carboxylic groups on the repeating unit of hyaluronic acid (HA) was added and the pH was readjusted to about 6.8. Next, 3 mmol of HOBt per mol of available carboxylic groups on the repeating unit of HA was added and the reaction solution was maintained at a pH of about 6.8 for about 9 hours. Subsequently, sodium chloride (NaCl) was dissolved at a concentration of 5 mg/mL in final reaction mixture. Precipitation in ethanol removed the unreacted reagents. A final precipitate was redissolved in DI water and dialyzed using a dialysis membrane with a 3-5 kDa molecular-weight cut-off (MWCO) against an aqueous solution containing 0.5 g/mL NaCl. Water was removed by freeze-drying to obtain the HA-g-dopamine final product.

Similar HA-g-dopamine conjugates were synthesized from HA having a weight average molecular weight of about 350 kDa, about 700 kDa, and about 1500 kDa.

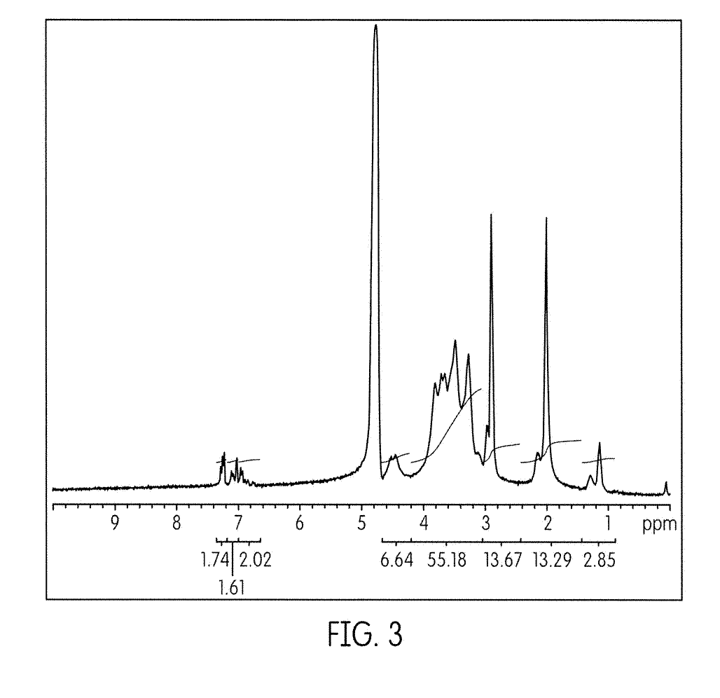

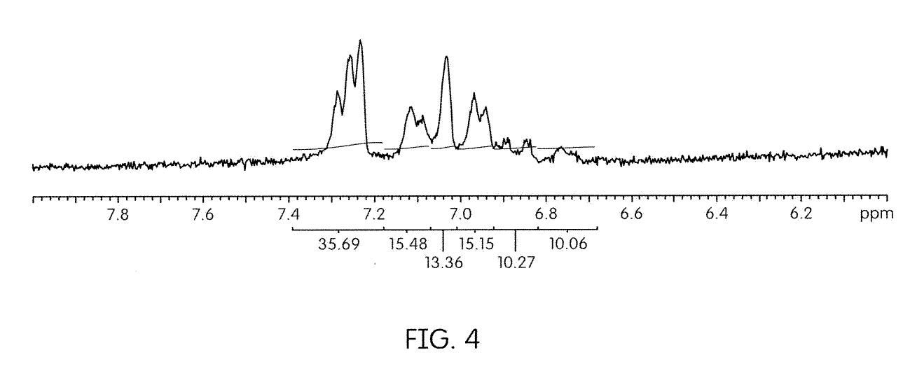

In one example, the obtained HA-g-dopamine was characterized by proton (¹H) nuclear magnetic resonance (NMR) spectroscopy. FIG. 3 shows the proton NMR spectrum obtained for the HA-g-dopamine in D₂O at a concentration of about 8-10 mg/mL. Referring to FIG. 4, the peak area for the peaks above 7 ppm being significantly greater than the peak area of the peaks below 7 ppm indicates that most of the dopamine present in the sample is conjugated to the HA, and only a minimal amount of unconjugated dopamine is present. The percentage of dopamine conjugation on the HA chains was estimated to be approximately 25% from the NMR spectrum, but it was difficult to calculate an exact percent of conjugation of dopamine due to the presence of some latent solvent in the freeze-dried sample of HA-g-dopamine. Changes in the NMR spectrum between a fresh sample and a two week old sample indicated that some degradation of the HA-g-dopamine was occurring over that time period.

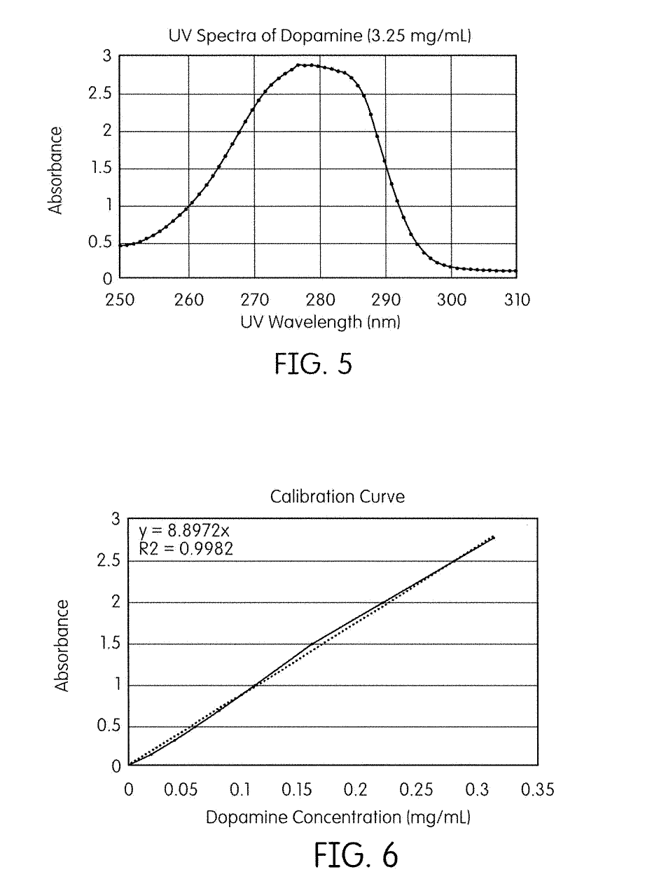

Because dopamine absorbs UV light at around 280 nm, as shown in FIG. 5, UV spectrum measurements were performed on the HA-g-dopamine using a UV-vis spectrophotometer and 1-cm quartz cells to determine more precisely the percent of dopamine conjugation. A calibration curve was obtained by measuring absorbance of dopamine solution as a function of its concentration, with the UV absorbance of dopamine at 280 nm being substantially linearly with respect to concentration in the range of 0-5 mg/mL, as shown in FIG. 6. Subsequently, the absorbance of 1 mg/mL HA-g-dopamine was observed and the percentage of conjugated dopamine was calculated based on the calibration curve of dopamine. In one example, the percent of dopamine conjugation on HA-g-dopamine was 22% as calculated using calibration curve of dopamine.

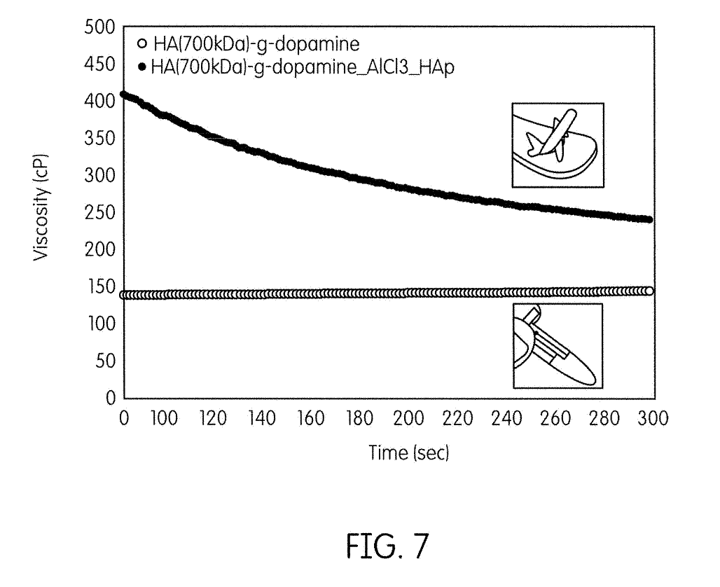

In one example, the viscosity of a 5 wt % solution of HA-g-dopamine in DI water was measured in a rheometer using cone-plate geometry at 37° C. at 10 Pa shear stress, where the HA had a weight average molecular weight of about 700 kDa. The viscosity of the solution was about 150 centipoise (cP), as shown in FIG. 7. A 150-μL, suspension of hydroxyapatite particles at a concentration in the range of 2-3 mg/mL in artificial saliva was mixed with 300 μL of the HA-g-dopamine solution and the viscosity was re-measured. In some embodiments, the concentration of the hydroxyapatite particles in artificial saliva is in the range of 1-100 mg/mL. The addition of the hydroxyapatite suspension caused the formation of a viscous gel with a viscosity of about 300-400 cP within about one minute. This viscosity slowly decreased over time to about 250 cP at about five minutes after the initial mixing, as shown in FIG. 7.

Performance Test: Adhesion of HA-g-Dopamine to Wet Bovine Teeth

In one example, the adhesion of HA-g-dopamine to wet bovine teeth was performed using a conventional protocol. Namely, a 1 wt % solution of HA-g-dopamine in DI water containing aluminum chloride (AlCl₃) was prepared. Bovine teeth were incubated in water at 37° C. overnight. The bovine teeth were removed from the water 30 seconds prior to applying the HA-g-dopamine solution to the bovine teeth, and the teeth were then dried for 2-3 minutes. The teeth were then incubated in water at 37° C. for 2 hours. An Alcian blue solution (1 wt % Alcian blue dye in acidic acid at a pH in the range of 3-5), which specifically stains certain polysaccharides including HA, was used to stain the bovine teeth to check the level of adhesion of the HA to the teeth. The teeth were dipped in the Alcian blue solution for 30 min at 37° C., extensively washed with water, and then photographed in color.

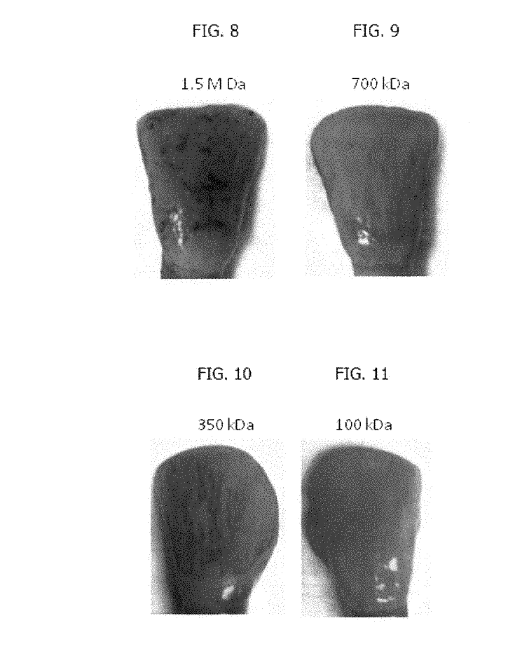

Referring to FIG. 8 through FIG. 11, the 2-hour adhesion process clearly demonstrated binding of the HA-g-dopamine with the enamel of the bovine teeth and the effect of the molecular weight of the HA on binding. FIG. 8 shows that for the highest molecular weight HA conjugated to dopamine, 1500 kDa, areas of very dark blue staining, areas of moderate staining, and areas of light blue staining were observed. As the molecular weight of the HA decreased from 1500 kDa (FIG. 8) to 700 kDa (FIG. 9) to 350 kDa (FIG. 10) to 100 kDa (FIG. 11), the staining became more uniform, as the size and level of more intensely stained areas decreased. FIG. 11 shows that the adhesion of the low molecular weight conjugate of HA-g-dopamine was highly uniform. Without being bound by theory, it is believed that the higher molecular weights lead to increased amounts of swollen polymer on the teeth, which stained darker than the less swollen polymer.

Although the HA-g-dopamine system showed good adhesion, the temperature sensitivity and levels of degradation of that system were greater than ideal for use in a dental varnish. Dopamine was conjugated to PAA, as an alternative to the HA-g-dopamine system, by using a similar protocol to the protocol used for the synthesis of HA-g-dopamine. Dopamine was conjugated to PAA by way of an amine bond formed between the amine group of the dopamine and a carboxylic acid group on the PAA using EDC/HOBt catalysis chemistry. In one embodiment, the percent conjugation of dopamine in the PAA-g-dopamine may be in the range of 5 to 80%. In some embodiments, the percent conjugation of dopamine in the PAA-g-dopamine is in the range of 20 to 30%.

### Example-2 Synthesis of PAA-g-Dopamine in the Presence of an Antioxidant

500 mg of polyacrylic acid (PAA) having a weight average molecular weight of about 450 kDa was dissolved in 150 mL of DI water at room temperature for 5 to 10 hours. The pH of the solution was then adjusted to about 6.8 with 0.1N HCl and 0.1N NaOH aqueous solutions. Ascorbic acid was then added to the solution as the antioxidant to achieve a concentration of 1 mg/mL. About 3 mmol of EDC per mol of available carboxylic groups on the repeating unit of polyacrylic acid was added, and the pH was readjusted to about 6.8. Next, 3 mmol of HOBt per mol of available carboxylic groups on the repeating unit of polyacrylic acid was added to the reaction mixture and a pH of about 6.8 was re-established. Subsequently, dopamine was added to the solution in a 30:100 molar ratio with respect to the number of available carboxylic groups on the repeating unit of PAA, and the solution was then maintained at a pH of about 6.8 for about 9 hours. Finally, the reaction mixture was dialyzed using a dialysis membrane with a 3-5 kDa MWCO against an aqueous solution containing 0.5 g/mL NaCl for two days and then against DI water for one day. Water was removed by freeze-drying to obtain the PAA-g-dopamine final product.

Synthesis of a control PAA-g-dopamine was repeated using above-described protocol without adding ascorbic acid during the synthesis.

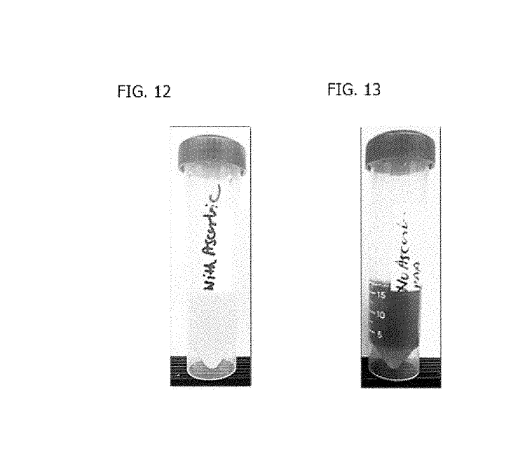

Referring to FIG. 12, the PAA-g-dopamine synthesized in the presence of ascorbic acid during the synthesis was colorless after the dialysis. Referring to FIG. 13, however, black coloration was observed in the PAA-g-dopamine that was synthesized in the absence of ascorbic acid. Quinone formation resulting from the oxidation of dopamine/catechol causes the black coloration. The presence of antioxidant molecules, such as, for example, ascorbic acid or sulfur dioxide, in the solution prevents the black coloration. Without wishing to be bound by theory, the prevention of oxidation is presumably due to either scavenging oxygen or reducing ortho-quinone derivatives formed from the oxidation of phenolic compounds.

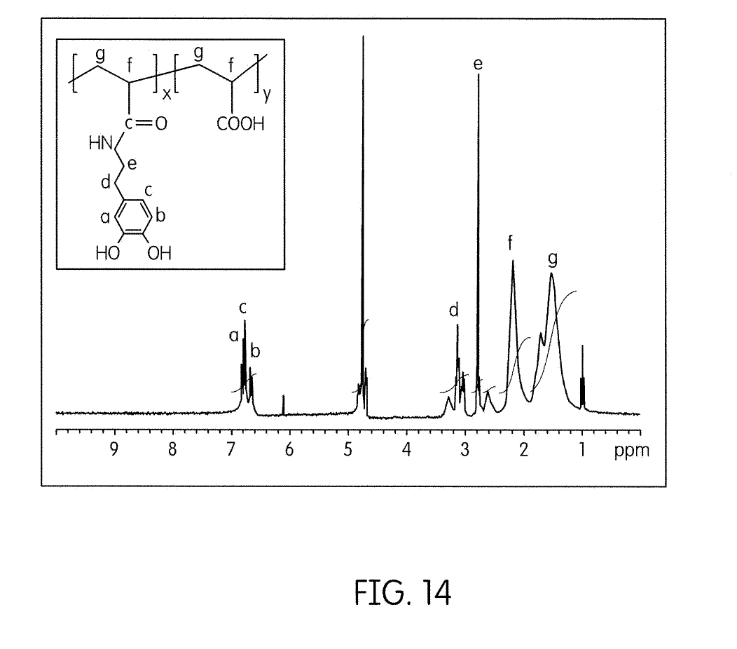

In one example, the obtained PAA-g-dopamine was characterized by proton (¹H) nuclear magnetic resonance (NMR) spectroscopy. FIG. 14 shows the proton NMR spectrum obtained for the PAA-g-dopamine in D₂O. In the NMR of PAA-g-dopamine, the peaks found at δ 4.79 ppm correspond to the deuterated water that was used for sample dissolving. The two peaks at δ 1.58-1.88 ppm (G) and 2.3 ppm (F) correspond to the hydrogens of the PAA polymeric backbone (—CH(CH₂—)— and —CH(CH₂—)—, respectively). The peaks found at δ 2.7-2.8 ppm (E) and 3.0-3.2 ppm (D) correspond to the hydrogens of —CH₂—CH₂— of the dopamine. The peaks found between δ 6.6 ppm and 6.8 ppm (A, B, C) correspond to the aromatic hydrogens of C₆H₃(OH)₂ of the dopamine. Thus, both PAA and dopamine were present in the sample. Furthermore, the dopamine grafting ration on PAA was estimated by the formula f=A/A₀. The amount of H in the aromatic rings of grafted dopamine molecules was represented as A through calculating the integral area of the peaks at δ 6.6-6.8 ppm. A₀ was the integral area of the peaks at δ 1.4-2.5 ppm representing the amount of H in the polymeric backbone. By this methodology, the percent conjugation was calculated to be approximately 13% and there was no change in this ratio between a fresh sample and a two-week-old sample, indicating no degradation over that time period.

Adhesion of PAA-g-Dopamine to Wet Bovine Teeth

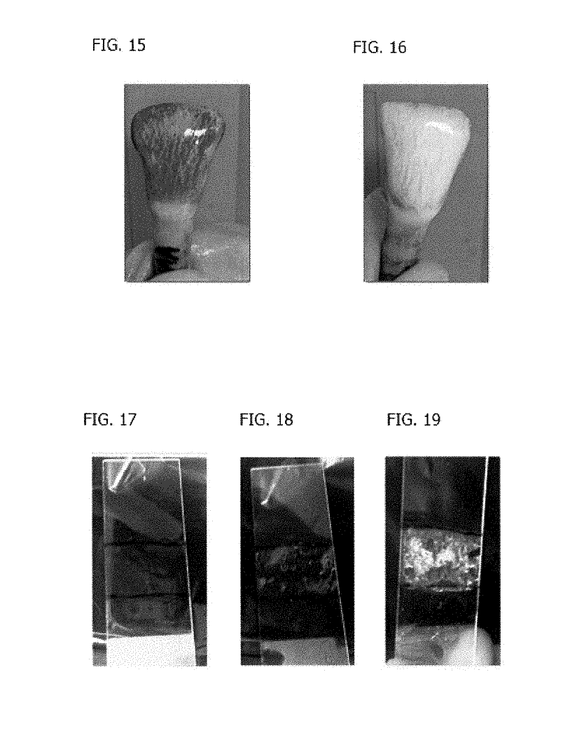

In one example, the adhesion of PAA-g-dopamine to wet bovine teeth was performed using a conventional protocol similar to that used for HA-g-dopamine. A 10 wt % solution of PAA-g-dopamine dissolved in DI water was painted on wet teeth. After 2 to 3 minutes, a varnish was applied to the painted teeth and similar teeth that were unpainted. The teeth were incubated in artificial saliva at 37° C. for about 2 hours. After the incubation, the teeth were each dipped in a 0.01 wt % aqueous neutral red solution, which stains PAA since neutral red is positively charged and PAA is negatively charged for 5 minutes at room temperature. The teeth were then washed with DI water and left overnight in DI water. Finally, the teeth were removed from the DI water and photographed. A comparison of the photograph of the painted tooth, as shown in FIG. 15, and the photograph of the unpainted tooth, as shown in FIG. 16, shows a much higher level of neutral red staining of the painted tooth, indicating adhesion of the PAA-g-dopamine to the bovine tooth.

Remineralization: Calcium Absorption by Hydrogel-Forming Polymer Formulation

In one example, a dental varnish composition promoted in situ biomimetic remineralization by being capable of absorbing calcium from surrounding saliva. About 200 mg of PAA-g-dopamine was dissolved in about 4.4 mL of DI water. Separately, a solution was formed by combining about 60 mg of ascorbic acid, about 20 mg of aluminum chloride in 0.5 mL DI water, and about 0.55 mL of 5 M NaOH and then combined with the PAA-g-dopamine solution. Next, about 100 mg of boric acid was dissolved in about 1 mL of DI water and added to solution. Finally, about 0.27 mL of 48-50 wt % hydrofluoric acid was added to the solution and the pH was adjusted to 8.0 using 5 M NaOH.

The prepared varnish formulation was applied to a glass slide, and the glass slide was incubated in a 25-mM calcium chloride solution for about 30 minutes. As a control, another glass slide with the prepared varnish formulation was incubated in DI water for about 30 minutes.

Referring to FIG. 17, the glass slide from the control was still transparent after 30 minutes of incubation. In contrast, the glass slide incubated in calcium chloride was slightly whitish in color after one minute, as shown in FIG. 18, and became more whitish upon full incubation for thirty minutes, as shown in FIG. 19, which clearly indicates the absorption of CaCl₂ by the varnish dipped in the CaCl₂ solution.

Preparation of Water Soluble Dental Composition

In one example, improved adhesion of a dental varnish to enamel was achieved by including stimulus moieties in the varnish formulation. First, about 175 mg of PAA-g-dopamine was dissolved in about 4.4 mL of DI water. Separately, about 60 mg of ascorbic acid and 20 mg of aluminum chloride were dissolved in about 0.5 mL of DI water, and then about 0.55 mg of a branched polyethylene imine having an average molecular weight of about 600 Da was added. After mixing thoroughly, the branched polyethylene imine solution was added to the PAA-g-dopamine solution. Then, about 100 mg boric acid was dissolved in about 1 mL DI water and added to the PAA-g-dopamine/branched polyethylene imine mixture. Next, another 0.55 mg of the branched polyethylene imine was added followed by 0.27 mL of hydrofluoric acid, to provide a fluoride source in the final stimulus-moiety-containing varnish formulation. The branched polyethylene imine provided the stimulus moieties for the varnish formulation.

A control varnish formulation was prepared for comparison by the same method as the stimulus-moiety-containing varnish formulation, except for replacing the branched polyethylene imine with 5 M NaOH.

Adhesion of Varnish Formulation to Enamel-Like Hydroxyapatite Discs

An adhesion test was then performed on enamel-like hydroxyapatite discs. Before the stimulus-moiety-containing varnish formulation or the control varnish formulation was applied, however, the hydroxyapatite discs were incubated in DI water for 1 to 2 hours. The prepared varnishes were then applied to the wet hydroxyapatite discs and were given about 2 to 3 minutes to bind to the discs. As a second control, a wet hydroxyapatite disc received no varnish formulation to confirm that the Alcian blue dye is absorbed by the varnish and not by the hydroxyapatite discs.

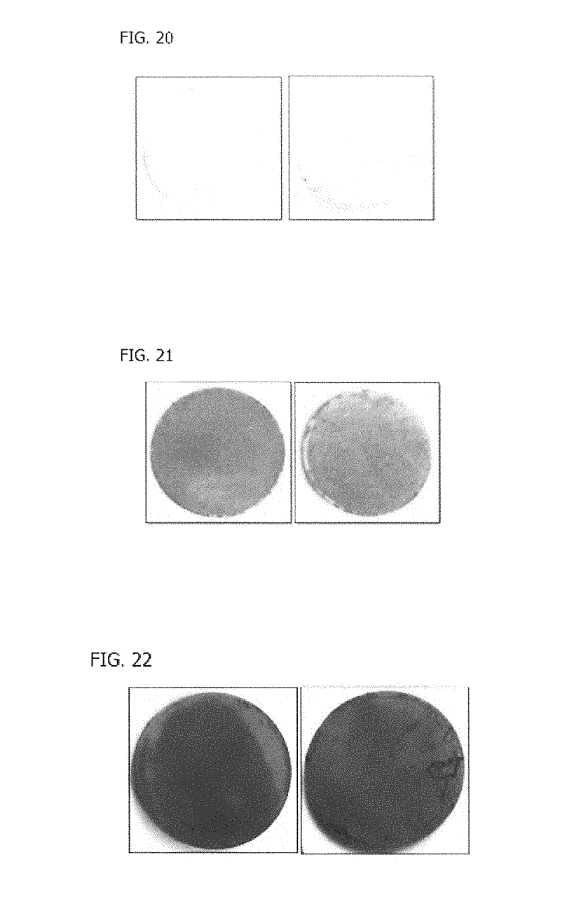

The three hydroxyapatite discs were then separately dipped in an artificial saliva solution. After 2 hours of incubation at about 37° C., the hydroxyapatite discs were washed with DI water and then air-dried. Finally, the hydroxyapatite discs were stained with Alcian blue dye (pH 1.5-2.0). Referring to FIG. 20, the hydroxyapatite disc with no varnish was nearly white with only some faint areas of blue staining. FIG. 21 shows that the hydroxyapatite disk with the control varnish was moderately stained by the blue dye to a light blue color. FIG. 22, however, shows strong staining of the hydroxyapatite disc with the stimulus-moiety-containing varnish to a deep, fairly uniform blue color, indicating higher adhesion to a hydroxyapatite disc when the varnish composition contains stimulus moieties.

Synthesis of Varnish Formulation for Biocompatibility, F Release and F Uptake Testing

Polyacrylic acid (1000 mg) was dissolved in PBS buffer (100 mL) at room temperature for 1 hr and pH was adjusted to 12.00 with 0.1N hydrochloric acid (HCl) and 0.1N sodium hydroxide (NaOH) aqueous solution. Then, EDC (1200 mg) was added and pH was adjusted to 9 and NHS (1200 mg) was added and pH was adjusted ˜6.2. Next, dopamine (984 mg) was added to reaction mixture, then maintained the pH of reaction mixture ˜6.1 for ˜2 hr. Then, ascorbic acid (200 mg) was added to reaction mixture and pH was adjusted around 6.1 and reaction was continued for 24 hr. After overnight, pH of solution was reduced to 4.00 using 0.1M NaOH and 0.1M HCl, and white precipitate of PAA-g-dopamine was settled and supernatant was discarded. The white precipitate was redissolved in DI water containing sodium tetraborate decahydrate solution (1 gm/17.5 mL) at pH 6.00. Precipitation step was repeated two times. Then, collected precipitate was mixed with 5 ml of aluminum fluoride solution 32 mg/1 ml) and 14 ml of sodium tetraborate decahydrate solution and add DI water to make total volume 50 mL, then mixed thoroughly until ingredient dissolved uniformly. In 20 ml of this solution, 2 ml polyethylene imine (PEI) 600 Da and 0.5 ml HF (48-51%) were mixed thoroughly, then 1.0 ml PEI 1200 Da and another 0.5 ml of HF (48-51%) were mixed. Finally, 1 ml aluminum chloride solution (20 mg/ml) was mixed and pH was adjusted 7.5-8.00 from prepared pH 10 using concentrated hydrochloric acid.

Biocompatibility

Quantitative MTT Cytotoxicity Assay was used for determining the cytotoxic response of extraction of varnish formulations using L-929 mammalian fibroblast cells. The assay measures viability of cells through metabolic activity, as the mitochondrial dehydrogenases of living cells convert the yellow MTT solution into blue-violet insoluble formazan. Formazan crystals are dissolved in isopropanol to make a homogeneous solution for photometric measurements. The number of viable cells correlates to the color intensity.

To collect extraction, 20 hydroxyapatite discs were used for preparation per article. 200 mg of each test article was applied to the 20 discs (10 mg per disc) and the discs were allowed to sit at room temperature for 5 minutes. The hydroxyapatite discs were then rinsed with 10 mL of media for ˜10 seconds to remove the excess varnish. The rinsed discs were then incubated in extraction media. The comparison article consisted of 1 unrinsed hydroxyapatite disc with no varnish applied. The controls were prepared aseptically according to ISO rations and were tested in parallel with the test article. The MTT Media+10% FBS was added to the articles and controls based on the extraction ratio. The test and control articles were extracted with continuous agitation on an orbital shaker. The test and control article extraction media were visually inspected immediately prior to and post extraction. The extracts were used for testing within 24 hours of incubation completion. After extraction, extracts were centrifuged at 3000 RPM for 5 minutes then used for creating dilutions for assessing cell viability using the following dilutions: 100%, 50%, 25% and 12.5%. Then, cytotoxicity of collected extracts was tested in compliance to the International Organization for Standardization (ISO) 10993-5: 2009 and British Standard European Norm ISO (BS EN ISO) 10993-5: 2009 (Tests for in vitro Cytotoxicity).

TABLE 1
Cytotoxicity study using MTT assay as per ISO 10993-12: 2012

| | | | Cytotoxicity | | | |
|---|---|---|---|---|---|---|
| | | | No dilution | ½ dilution of | ¼ dilution of | ⅛ dilution of |
| Sample | Antioxidant | pH | (100% extract) | 100% extract | 100% extract | 100% extract |
| DHGV 27 | Borax | 7.5 | Toxic | Toxic | Toxic | Biocompatible |
| | | | (1.76% viability) | (43.45% viability) | (54.27% viability) | (82.06% viability) |
| DHGV 28 | Borax | 7.75 | Toxic | Biocompatible | Biocompatible | Biocompatible |
| | | | (52.24% viability) | (73.26% viability) | (75.02% viability) | (74.14% viability) |
| DHGV 29 | Borax | 8.00 | Toxic | Toxic | Biocompatible | Biocompatible |
| | | | (1.19% viability) | (51.31% viability) | (76.21% viability) | (111.85% viability) |
| DHGV 30 | Borax Buffer | 7.75 | Biocompatible | Biocompatible | Biocompatible | Biocompatible |
| | | | (83.74% viability) | (81.80% viability) | (81.88% viability) | (93.82% viability) |
| DHGV 31 | Cystiene | 7.65 | Toxic | Toxic | Toxic | Toxic |
| | | | (7.54% viability) | (3.64% viability) | (31.24% viability) | (56.05% viability) |
| DHGV 32 | Sodium | 7.75 | Toxic | Toxic | Toxic | Toxic |
| | Metasulfite | | (12.53% viability) | (55.29% viability) | (41.57% viability) | (52.41% viability) |

Fluoride Uptake by Enamel

Three formulations of varnish (DHGV 30, DHGV 32 and DHGV 36) were synthesized by varying the ingredient of varnish and tested for F uptake. For fluoride uptake experiment, sound bovine incisor enamel was embedded in the end of a plexiglass rod (¼″ diameter×2″ long) using methylmethacrylate. Subsequently, an artificial incipient lesion was formed in them by immersion into an about 0.1M lactic acid/0.2% Carbopol 907 50% saturated with calcium phosphate solution at about pH 5.0 for about 24 hours at about room temperature. The specimens were kept hydrated and stored at 40 C until time of use.

The 8 specimens per group were numbered and placed into a neoprene stopper with the enamel surface of the specimens being flush with the stopper. The stoppers have been specifically designed to evenly distribute the 8 enamel specimens around the outer edge of the stopper. A single layer of test varnish (approx. 0.0050±0.001 g) was applied to the surface of each individual specimen. The stopper was place in a specimen cup, enamel surfaces facing up. Tubing from the solution container (Artificial Saliva, see Appendix) passed through a multi-channel peristaltic pump and was affixed to a hole in the lid of the specimen cup. The multi-channel pump was set to provide a slow drip of solution (approximately 1.0 ml/min) centrally over the stopper (drip of solution did not fall directly onto any of the 8 specimens). The solution collecting on the surface (evenly covering all 8 specimens) eventually broke the tension holding it on the stopper and ran off into the bottom of the specimen cup. The specimen cup was equipped with a drain to ensure the solution level never reached the surface of the stopper. Therefore, the solution in contact with the varnish treated enamel specimens was slowly replaced by fresh solution, mimicking intra-oral salivary flow.

Following a 2-hour treatment time, the specimens were removed from the stopper and excess varnish was carefully removed (physical removal using a spatula and subsequent removal using a cotton swab saturated with reagent grade ethyl alcohol). The specimens were then rinsed well under running DI water for 30 seconds. One layer of enamel was removed from each specimen by immersion in 0.5 ml of 1.0 N perchloric acid (HClO₄) for 15 seconds. A sample of each solution was buffered with TISABII to a pH of 5.2 (0.25 ml sample, 0.5 ml TISABII and 0.25 ml 1N NaOH) and the fluoride content determined using a fluoride specific electrode by comparison to a similarly prepared fluoride standard curve. A second sample was analyzed via atomic absorption for calcium content for use in depth determination (0.05 ml sample diluted to 5.0 ml).

### Result:

DHGV 30 supported the highest F uptake (4910±154) compare to DHGV 32 (2776±223) and DHGV 36 (2626±140).

F Release Experiment

For each test sample, a minimum of two (2) replicates were prepared. Known amount of varnish formulations were applied to glass slides and varnish painted glass slides were transferred to container containing 10 ml artificial saliva. F release from the varnishes was allowed for 2 hours at room temperature. After two (2) hours, 10 ml of artificial saliva was transferred into a small plastic beaker containing 10 ml of TISAB II and both solutions were mixed to determine the release F ions in the solution.

TABLE 2
Fluoride release from varnish

| | | | | F (ppm) | | F |
|---|---|---|---|---|---|---|
| Sample | Antioxidant | AlCl₃ | AlF₃ | in Varnish | pH | Release |
| DHGV | Boric acid Buffer | ✓ | ✓ | 22600 | 7.75 | 19973 |
| 30 | | | | | | |
| DHGV | Boric acid Buffer + | ✓ | ✓ | 22600 | 7.75 | 20334 |
| 31 | L-cystiene | | | | | |
| DHGV | Boric acid Buffer + | ✓ | ✓ | 22600 | 7.75 | 22758 |
| 32 | Sodium MetaSulfite | | | | | |
| DHGV | Boric acid Buffer + | ✓ | ✓ | 11300 | 7.75 | 10519 |
| 33 | L-cystiene | | | | | |
| DHGV | Boric acid Buffer + | ✓ | x | 22600 | 7.75 | 21020 |
| 34 | L-cystiene | | | | | |
| DHGV | Boric acid Buffer + | x | ✓ | 22600 | 7.75 | 20500 |
| 35 | L-cystiene | | | | | |
| DHGV | Boric acid Buffer + | x | x | 22600 | 7.75 | 19975 |
| 36 | L-cystiene | | | | | |

Table 3 shows a comparison of certain components and properties of certain commercial varnishes to a water-soluble hydrogel-based dental varnish.

TABLE 3
Comparison of Available Varnish with Hydrogel-Based Varnish

| Feature | Nupro White | Fluor Protector | CleanPro | Duraphat | Hydrogel |
|---|---|---|---|---|---|
| Natural Resin | Hydrogenated | | Modified | Mastic or | Chitosan or |
| | rosin | | rosin | shellac | modified |
| | | | | | cellulose |
| Synthetic | Urethane | Polyurethane | | | Hyaluronic |
| Polymer | resin | | | | acid |
| Solvent | Isopropanol | Ethyl acetate/ | Ethanol and | Ethanol | Water |
| | | isoamyl | 20% water | | |
| | | propionate | | | |
| By-product | Yes | Yes | Yes | Yes | No |
| (e.g. polyurea) | | | | | |
| Bioactive | No | No | No | No | Yes |
| Polymer | | | | | |
| Viscosity | Low | Low | High | High | Low |
| Adhesion to | Physical | Physical | Physical | Physical | Covalent |
| Enamel | | | | | |
| Sensitization/ | Yes | Yes | Yes | Yes | No |
| Irritation | | | | | |
| Fluoride Type | NaF | Difluorosilane | NaF | NaF | NaF, Amino |
| Fluoride Percent | 5 wt % | 0.1 wt % | 5 wt % | 5 wt % | 5 wt % |
| Fluoride Form | Suspended | Suspended | Suspended | Suspended | Dissolved |
| Fluoride Release | Rapid | Slow | Slow | Slow | Rapid |
| Fluoride Uptake | Moderate | High | Low | Moderate | High |
| Mixing | Required | Required | Required | Required | Not required |
| Tooth Drying | Not required | Required | Not required | Required | Not required |

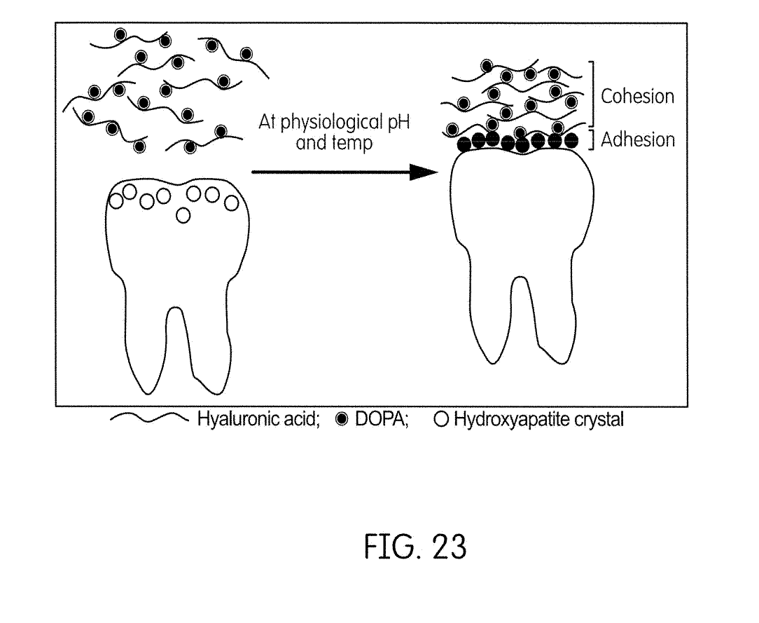

In some embodiments, a method includes applying a water-soluble hydrogel-based dental composition to a surface of a tooth, as shown in FIG. 23. The water-soluble hydrogel-based dental composition includes a hydrogel-forming polymer having cohesive properties and adhesive properties to dental enamel, a fluoride agent, and water. When the water-soluble hydrogel-based dental composition is applied to the surface of the tooth at a physiological temperature and pH, the hydrogel-forming polymer may simultaneously adhere to the enamel of the tooth and cohere to itself. More specifically, the hydrogel-forming polymer adheres to the hydroxyapatite of the tooth enamel. Since the water-soluble hydrogel-based dental composition is a water-based system in which fluoride salts are easily dissolved, the water-soluble hydrogel-based dental composition may be applied without any stirring, mixing, or shaking of the water-soluble hydrogel-based dental composition prior to application to a tooth.

In some embodiments, the adhesive and cohesive properties of the conjugate are pH-sensitive. In some embodiments, a method of applying the water-soluble hydrogel-based dental varnish to a tooth at physiological pH includes adjusting the pH at which the water-soluble hydrogel-based dental varnish is prepared, to an application pH, at which the level of cohesion of the water-soluble hydrogel-based dental varnish to itself and level of adhesion of the water-soluble hydrogel-based dental varnish to the tooth surface is higher.

In one embodiment, the pH at which the water-soluble hydrogel-based dental varnish is prepared may be about 10.

In another embodiment, the application pH may be from about 7.5 to about 8.5.

While the present disclosure has been described with reference to one or more embodiments, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims. In addition, all numerical values identified in the detailed description shall be interpreted as though the precise and approximate values are both expressly identified.

1. A composition comprising:
a hydrogel-forming polymer having cohesive properties to itself and adhesive properties to dental enamel, and
water;
wherein the composition does not include any ethanol, iso propanol, ethyl acetate, butyl acetate, isoamyl propionate or hexane.

2. The composition of claim 1, wherein the hydrogel-forming polymer comprises a water-soluble polymer and an adhesion promoter chemically and/or physically conjugated to the water-soluble polymer.

3. The composition of claim 2, wherein the hydrogel-forming polymer further includes at least one antioxidant.

4. The composition of claim 2, wherein the adhesion promoter provides the hydrogel-forming polymer with cohesive properties to itself and adhesive properties to dental enamel.

5. The composition of claim 2, wherein the adhesion promoter is selected from the group consisting of dopamine, dopamine with a conjugated electron-withdrawing group conjugated at the 6-position on the dopamine aromatic ring, dopamine complexed to an electron-withdrawing group at the hydroxyl groups of the dopamine, gallic acid, caffeic acid, ferulic acid, protocatechuic acid, coumaric acid, ellagic acid, resveratrol, rosmarinic acid, quercetin, and combinations thereof.

6. The composition of claim 5, wherein the conjugated electron-withdrawing group is a nitro group (—NO₂), a chloro group (—Cl), or a fluoro group (—F).

7. The composition of claim 5, wherein the complexed electron-withdrawing group is a borate or a borate derivative.

8. The composition of claim 5, wherein the adhesion promoter is dopamine.

9. The composition of claim 2, wherein the water-soluble polymer is selected from the group consisting of hyaluronic acid (HA), polyacrylic acid (PAA), chitosan, hydroxypropyl methylcellulose (HPMC), a water-soluble polyethylene glycol (PEG)-modified polymer, a water-soluble PEG-crosslinked polymer, a bis-thiol PEG, a water-soluble or partially water-soluble modified rosin, and combinations thereof.

10. The composition of claim 3, wherein the antioxidant is present in the hydrogel-forming polymer in amounts of from 0.1 to 14 mmol based on per mol of available functional groups on a repeating unit of the water-soluble polymer.

11. The composition of claim 3, wherein the antioxidant is ascorbic acid, sodium metabisulfite, boric acid, sodium tetraborate, 4,4′-Biphenyldiboronic acid, benzene-1,4-diboronic acid, 2,5-thiophenediyl bisboronic acid, sulfur dioxide, uric acid, tocopherol, or mixtures thereof.

12. The composition of claim 1, further comprising a metal ion source.

13. The composition of claim 12, wherein the metal ion source is selected from the group consisting of a divalent metal ion source, a trivalent metal ion source, and mixtures thereof.

14. The composition of claim 13, wherein the divalent metal ion source is selected from the group consisting of a salt of calcium, salt of zinc, salt of magnesium, salt of tin, salt of strontium, salt of chromium, salt of manganese, salt of beryllium, salt of barium, salt of cobalt, salt of nickel, salt of lead and salt of copper.

15. The composition of claim 13, wherein the trivalent ion source is selected from the group consisting of a salt of aluminum, salt of iron, salt of chromium, salt of bismuth, salt of manganese, salt of cobalt and salt of indium.

16. The composition of claim 1, further comprising at least one stimulus moiety.

17. The composition of claim 16, wherein the at least one stimulus moiety is a branched cationic polymer.

18. The composition of claim 17, wherein the branched cationic polymer is selected from the group consisting of a polylysine, a polyarginine, branched polyethyleneimine, branched poly(diallyldimethylammonium chloride), and combinations thereof.

19. The composition of claim 17, wherein the branched cationic polymer is selected to increase an adhesion kinetic between the composition and dental enamel.

20. A water soluble dental varnish composition comprising (a) a hydrogel-forming polymer having cohesive properties and adhesive properties to a dental enamel, and
(b) a fluoride agent;
wherein the hydrogel-forming polymer comprising a water-soluble polymer and dopamine chemically and/or physically conjugated to the water-soluble polymer.

21. The water soluble dental varnish composition according to claim 20, wherein the conjugated dopamine on the water soluble polymer adheres to any calcium ion present on the enamel surface and also absorbs calcium ions from a surrounding medium to the hydrogel-forming polymer.

22. The water soluble dental varnish composition according to claim 20, wherein the hydrogel-forming polymer further includes at least one antioxidant.

23. The water soluble dental varnish composition according to claim 20, further comprising a metal ion source.

24. The water soluble dental varnish composition according to claim 20, further comprising one or more of a thickener, a tackifier, a flavoring, a sweetner, a colorant or a combination thereof.

25. The water soluble dental varnish composition according to claim 20, further comprising a stimulus moiety.

26. The water soluble dental varnish composition according to claim 25, wherein the stimulus moiety is a branched cationic polymer.

27. The water soluble dental varnish composition according to claim 26, wherein the branched cationic polymer is selected from the group consisting of a polylysine, a polyarginine, branched polyethyleneimine, branched poly(diallyldimethylammonium chloride), and combinations thereof.

28. The water soluble dental varnish composition according to claim 20, wherein the fluoride agent is selected from the group consisting of sodium fluoride, stannous fluoride, acidulated phosphate fluoride, amine fluoride, fluorosilane, and mixture thereof.

29. The water soluble dental varnish composition according to claim 28, wherein the amine fluoride is selected from the group consisting of N′,N′-tri-(polyoxyethylene)-N-hexadecylpropylene diamine dihydrofluoride; 9-octadecylamine hydrofluoride, hexadecylamine hydrofluoride, and bis-(hydroxyethyl)-aminopropyl-N-hydroxyethyloctadecylamine dihydrofluoride.

30. The water soluble dental varnish composition according to claim 20, wherein the hydrogel-forming polymer is present in a concentration of from 0.01 weight percent to 50 weight percent based on a total volume of the composition.

31. The water soluble dental varnish composition according to claim 20, wherein the stimulus moiety is present in a concentration of from 0.1 to 50 weight percent based on a total volume of the composition.

32. The water soluble dental varnish composition according to claim 20, wherein the fluoride agent is present in a concentration of from about 0.01 to about 10 weight percent based on a total weight of the composition.

33. The water soluble dental varnish composition according to claim 20, wherein the dental varnish release fluoride ions in a concentration from about 1000 ppm to about 22600 ppm.

34. A method of preparing a water soluble dental composition; said method comprising:
(a) dissolving a hydrogel-forming polymer in water to form a hydrogel-forming polymer solution;
(b) dissolving an antioxidant and a metal ion source in water to form a first solution and adding a branched cationic polymer to the first solution to form a branched cationic polymer solution;
(c) adding the branched cationic polymer solution to the hydrogel-forming polymer solution to prepare a hydrogel-forming polymer/branched cationic polymer mixture;
(d) dissolving an antioxidant in water to form a second solution and adding the second solution to the hydrogel-forming polymer/branched cationic polymer mixture to form an antioxidant polymer mixture;
(e) mixing the antioxidant polymer mixture with a fluoride agent to form the water soluble dental composition.
wherein the branched cationic polymer is selected to increase an adhesion kinetic between the composition and dental enamel.

35. The method according to claim 34, wherein the hydrogel-forming polymer is present in the water soluble dental composition in amounts of from about 0.01 to about 50 weight percent based on a total volume of the composition.

36. The method according to claim 34, wherein the antioxidant is present in the water soluble dental composition in amounts of from about 0.005 mmole/ml to about 20 mmole/ml based on a total volume of the hydrogel-forming polymer.

37. The method according to claim 34, wherein the metal ion source is present in the water soluble dental composition in amounts of from about 0.001 mmole/ml to about 5 mmole/ml based on a total volume of the hydrogel-forming polymer.

38. The method according to claim 34, wherein the fluoride agent is present in the water soluble dental composition in amounts of from about 1000 ppm to about 22600 ppm based on a total volume of the composition.

39. The method according to claim 34, wherein the branched cationic polymer is present in the water soluble dental composition in amounts of from about 0.1 weight percent to about 50 weight percent based on a total volume of the composition.

40. A method comprising applying a water-soluble dental composition to a surface of a tooth at a physiological temperature and pH, wherein the water-soluble dental composition comprises a hydrogel-forming polymer having cohesive properties to itself and adhesive properties to dental enamel, a fluoride agent, and water.

41. The method according to claim 40, wherein the hydrogel-forming polymer adheres to the hydroxyapatite of tooth enamel, and coheres to itself.

42. The method according to claim 40, wherein the method does not require any stirring, mixing or shaking of the composition prior to application to a tooth.

43. The method according to claim 40, wherein the method includes adjusting the pH at which the water-soluble dental composition is prepared, to an application pH, at which the level of cohesion of the water-soluble hydrogel-based dental varnish to itself and level of adhesion of the water-soluble hydrogel-based dental varnish to the tooth surface is higher.

44. The method according to claim 43, wherein the pH at which the water-soluble dental composition is prepared is about 10.

45. The method according to claim 43, wherein the application pH is from about 7.5 to 8.5.
